(19) United States
(12) Patent Application Publication
(10) Pub. No.: US 2010/0222426 A1
(43) Pub. Date: Sep. 2, 2010

(54) Anti-Bacterial Agents

(76) Inventors: Simon Gibbons, London (GB); John P. Malkinson, London (GB); Winnie Shiu, Middlesex (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/677,912

(22) PCT Filed: Sep. 15, 2008
(86) PCT No.: PCT/GB08/03106

§ 371 Date: Mar. 12, 2010

(30) Foreign Application Priority Data
Sep. 14, 2007 (GB) 0718023.5

(51) Int. Cl. A61K 31/335; C07C 49/657; C07D 303/34; A61K 31/12; A61P 31/04

(52) U.S. Cl. 514/475; 568/308; 549/554; 514/678

(57) ABSTRACT

The invention relates to novel acylphloroglucinols which have strong growth inhibitory effects on multi-drug resistant strains of bacteria, particularly MRSA. Typically the compounds have a terpene substituent, or a terpene-derived substituent. Methods of isolating the compounds from natural sources, and synthetic methods for forming the compounds are also provided.

### Field of the Invention

The present invention relates to a novel set of compounds which have anti-bacterial activity. The compounds are derived from plant natural products and display activity against bacteria such as methicillin-resistant Staphylococcus aureus and multidrug-resistant S. aureus.

### BACKGROUND TO THE INVENTION

Staphylococcus aureus is a commensal organism that is a major hospital pathogen. Strains of this species that are resistant to β-lactams, notably the methicillin-resistant Staphylococcus aureus (MRSA) strains have been described from clinical sources for over 40 years. It is the ability of this Gram-positive organism to acquire resistance to practically all useful antibiotics that has become cause for considerable concern. The threat of untreatable multi-drug resistant bacteria has prompted a special report from the House of Lords and a report on hospital acquired infections by the National Audit Office in the UK. The latter estimates that hospital acquired infections and treatment of drug resistant bacteria in the clinical setting cost the taxpayer an estimated one billion pounds per annum.

The occurrence of a fully vancomycin-resistant strain of MRSA in the US in 2002 shows that the successful treatment of MRSA strains using vancomycin is not guaranteed. Linezolid (Zyvox™), a new member of the oxazolidinone group and the streptogramin quinupristin/dalfopristin mixture (Synercid™) are recently developed anti-staphylococcal agents and have been heralded as a solution to MRSA infections. However, an isolated report of resistance to linezolid in a clinical isolate of Staphylococcus aureus demonstrates that researchers should be not be complacent in this area, and that there is a continual need for a pipeline of new agents to combat multidrug-resistant bacteria.

The review in Nat. Prod. Rep., 2004, 21,263-277 describes plant-derived anti-staphylococcal compounds which act as either bacterial resistance modifying agents or have direct anti-bacterial action. The review discusses natural products such as monoterpenes, sesquiterpenes, simple phenols, flavonoids, alkaloids and polyketides. Acylphloroglucinols such as the natural product found in hyperforin are also described. Hyperforin comes from St John's Wort (Hypericum perforatum) and has Minimum Inhibitory Concentration (MIC) values of 0.1 to 0.5 μg/ml against MRSA and penicillin-resistant Staphylococcus aureus. However, this metabolite has a number of drawbacks. For instance, it is a chemically complex molecule and is therefore a very difficult target in terms of chemical synthesis. The molecule is also unstable, and has to be stabilised by the trapping of the molecule as a salt. It can also undergo tautomerism which contributes to the difficulty of its analysis and stability.

During the American Society of Pharmacognosy Annual Meeting in 2006, the inventors presented a poster which described an anti-staphylococcal Hypericum genus. Large scale collection and extraction from Hypericum beanii was carried out. The poster described a bioassay-guided fractionation of the dichloromethane extract which afforded a new acylphloroglucinol, 1,1,5-dihydroxy-2-(2′-methylpropionyl)-3-methoxy-6-methylbenzene. The structure of this compound is shown below.

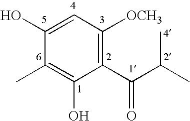

A related acylphloroglucinol, 1,5-dihydroxy-2-(2′-methybutanoyl)-3-methoxy-6-methylbenzene was also isolated, together with the known compound 1,7-dihydroxyxanthone. The structures of these compounds were characterised by extensive 1- and 2D NMR spectroscopy and mass spectroscopy. The minimum inhibitory concentration values of the acylphloroglucinol mixture and 1,7-dihydroxyxanthone against a panel of multidrug-resistant strains of Staphylococcus aureus ranged from 16-32 μg/ml and 128-256 μg/ml respectively.

In view of the above there remains a need to provide improved acylphloroglucinols which have stronger inhibitory effects on multidrug-resistant strains of bacteria, particularly multidrug-resistant strains such as MRSA.

### SUMMARY OF THE INVENTION

In accordance with a first aspect of the invention, there is provided a compound of general formula (I)

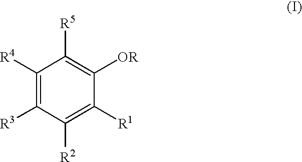

wherein R is selected from C₁₋₂₀ alkyl, C₂₋₂₀ alkoxyalkyl, C₂₋₂₀ alkenyl, C₂₋₂₀ alkynyl, C₃₋₂₀ cycloalkyl and C₄₋₂₀ (cycloalkyl)alkyl;

R¹ is C(O)R⁸;

R² and R⁴ are each independently selected from OR⁷;

R³ and R⁵ are each independently selected from H, —C≡C—H, halo, NH₂, NHR⁶, N(C₁₋₆ alkyl)₂, NHC(O)R⁶, NO₂, CN, C(O)H, C₁₋₁₀ alkyl, C₂₋₁₀ alkoxyalkyl, C₇₋₂₀ alkoxyaryl, C₁₂₋₂₀ aryloxyaryl, C₇₋₂₀ aryloxyalkyl, C₁₋₁₀ alkoxy, C₆₋₂₀ aryloxy, C₂₋₁₀ alkenyl, C₂₋₁₀ alkynyl, C₃₋₂₀ cycloalkyl, C₄₋₂₀ (cycloalkyl)alkyl, C₇₋₂₀ aralkyl, C₇₋₂₀ alkaryl, C₁₋₂₀ heteroaryl and C₆₋₂₀ aryl;

wherein R⁶ is selected from C₆₋₂₀ aryl, C₇₋₂₀ alkaryl, C₇₋₂₀ aralkyl, C₁₋₆ alkyl, C₁₋₂₀ heteroaryl, C₂₋₂₀ heterocyclyl, C₂₋₂₀ heteroaralkyl, C₃₋₂₀ heterocyclylalkyl, C₃₋₂₀ alkylheterocyclyl, C₆₋₂₀ arylamino and C₂₋₂₀ heteroarylamino;
wherein each R⁷ may be the same or different and is selected from C₁₋₂₀ alkyl, H, C₂₋₁₀ alkoxyalkyl, C₇₋₂₀ alkoxyaryl, C₁₂₋₂₀ aryloxyaryl, C₇₋₂₀ aryloxyalkyl, C₂₋₁₀ alkenyl, C₂₋₁₀ alkynyl, C₃₋₂₀ cycloalkyl, C₄₋₂₀ (cycloalkyl)alkyl, C₇₋₂₀ aralkyl, C₇₋₂₀ alkaryl, C₁₋₂₀ heteroaryl, C₆₋₂₀ aryl, C(O)H, C(O)OH, C(O)R⁸, C(O)OR⁶, C(O)NH₂, C(O)NR⁶H and C(O)N(C₁₋₆ alkyl)₂; and

wherein R⁸ is C₁₋₂₀ alkyl, H, OH, halo, NH₂, C₂₋₁₀ alkoxyalkyl, C₇₋₂₀ alkoxyaryl, C₁₂₋₂₀ aryloxyaryl, C₇₋₂₀ aryloxyalkyl, C₁₋₁₀ alkoxy, C₆₋₂₀ aryloxy, C₂₋₁₀ alkenyl, C₂₋₁₀ alkynyl, C₃₋₂₀ cycloalkyl, C₄₋₂₀ (cycloalkyl)alkyl, C₇₋₂₀ aralkyl, C₇₋₂₀ alkaryl, C₁₋₂₀ heteroary, C₆₋₂₀ aryl, OR⁶, NHR⁶ or N(C₁₋₆ alkyl)₂;

wherein any of the groups alkyl, aryl, alkaryl, aralkyl, heteroaryl, heterocyclyl, heteroaralkyl and heterocycloalkyl may be independently substituted on the backbone with one or more of the groups, preferably 1, 2, 3, 4, 5 or 6 groups, independently selected from C(O)OH, C(O)O(C₁₋₆ alkyl), C(O)O(C₆₋₂₀ aryl), C(O)O(C₇₋₂₀ aralkyl), C(O)O(C₇₋₂₀ alkaryl), NHC(O)—CH═CH₂, —C≡C—H, halo, OH, O(C₁₋₆ alkyl), O(C₆₋₂₀ aryl), O(C₇₋₂₀ alkaryl), O(C₇₋₂₀ aralkyl), ═O, NH₂, ═NH, NH(C₁₋₆ alkyl), N(C₁₋₆ alkyl)₂, ═N(C₁₋₆ alkyl), NH(C₆₋₂₀ aryl), NH(C₇₋₂₀ alkaryl), NH(C₇₋₂₀ aralkyl), NHC(O)(C₁₋₆ alkyl), NHC(O)(C₆₋₂₀ aryl), NHC(O)(C₇₋₂₀ alkaryl), NHC(O)(C₇₋₂₀ aralkyl), NHC(O)(C₁₋₂₀ heteroaryl), NHC(O)(C₂₋₂₀ heterocyclyl), NHC(O)(C₂₋₂₀ heteroaralkyl), NHC(O)(C₃₋₂₀ heterocyclylalkyl), NHC(O)(C₃₋₂₀ alkylheterocyclyl), NO₂, CN, C(O)H, C(O)(C₁₋₆ alkyl), C(O)(C₆₋₂₀ aryl), C(O)(C₇₋₂₀ alkaryl), C(O)(C₇₋₂₀ aralkyl), C₁₋₁₀ alkyl, C₂₋₁₀ alkoxyalkyl, C₇₋₂₀ alkoxyaryl, C₁₂₋₂₀ aryloxyaryl, C₇₋₂₀ aryloxyalkyl, C₁₋₁₀ alkoxy, C₆₋₂₀ aryloxy, C₂₋₁₀ alkenyl, C₂₋₁₀ alkynyl, C₃₋₂₀ cycloalkyl, C₄₋₂₀ (cycloalkyl)alkyl, C₇₋₂₀ aralkyl, C₇₋₂₀ alkaryl, C₁₋₂₀ heteroaryl, epoxide and C₆₋₂₀ aryl.

In accordance with the second aspect of the invention there is provided a pharmaceutical composition comprising a compound according to the first aspect of this invention and one or more pharmaceutically acceptable excipients.

In accordance with the third aspect of the invention there is provided a compound according to the first aspect of this invention for use in therapy, more particularly for use as an antibacterial in therapy.

A fourth aspect of this invention provides use of a compound according to the first aspect of this invention in the manufacture of a medicament for use in the treatment of Gram positive bacterial infections, more particularly Staphylococcus aureus infections, for instance MRSA, penicillin resistant, Mycobacterium tuberculosis resistant or multidrug-resistant S. aureus infections.

In accordance with a fifth aspect of the invention, there is provided a method of isolating the compound according to the first aspect of this invention from a plant comprising pulverising the plant substance suspected of containing the compound with one or more organic solvents to form one or more plant extracts, and purifying the extracts to obtain the compound.

In accordance with the final aspect of the invention there is provided a method for synthesising a compound according to the first aspect of this invention comprising reacting a compound of general formula (III)

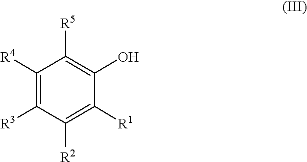

wherein R¹—R⁵ are as defined above;
with a compound of general formula (IV), which is R—Y, wherein R is as defined in claim 1 and Y is halo selected from Cl, Br or I, to give a compound of general formula (I).

The novel compounds of this invention have been shown to have greater activity than the compounds of the prior art against Gram-positive strains, in particular MRSA and penicillin resistant variant strains. In the assays performed by the inventors, the compounds have been found to have considerable activity against all multidrug-resistant strains tested, including those resistant to β-lactams, macrolides, fluoroquinolones and tetracyclines.

The novel compounds typically have at least one terpene substituent, or a terpene-derived substituent. Terpenes have multiples of five carbon atoms. Dervatives of terpenes are known as terpenoids. The compound is typically a monoterpenoid, sequiterpenoid or a diterpenoid. Terpenoids are widely found in nature. For instance, both human sex and adrenal hormones are steroidal and have terpene origin. The terpene substituent is thought to contribute to the efficacy of the novel compounds of this invention against drug-resistant bacteria.

The novel compounds according to the first aspect of this invention are stable and typically cannot undergo keto-enol tautomerism, since the preferred compounds are based on stable 1,3,5 tri-oxygenated benzene. Their synthesis is simple and may be achieved in as little as three steps, which makes the novel compounds commercially viable targets.

### DETAILED DESCRIPTION OF THE INVENTION

In the compound of general formula (I) R is typically C₁₋₂₀ alkyl, more preferably C₂₋₂₀ alkyl, for instance C₂₋₁₀ alkyl. R may be a C₃₋₂₀ straight or branched chain alkyl, preferably having 5 or more carbon atoms.

In a different embodiment, R is a group of general formula (II).

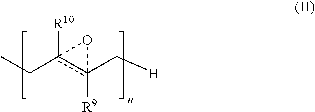

In the group of formula (II); the dotted lines means that the group is either saturated:

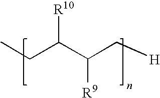

unsaturated:

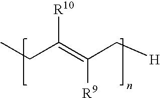

or contains an epoxide group:

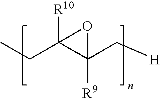

In this group, R⁹ and R¹⁰ are each independently H or C alkyl and n is 1-5.

Preferably, R⁹ is methyl. In typical compounds of the invention, R¹⁰ is H.

Each repeat unit of the group within the bracket need not be the same in the group of formula (II). Thus, the group may be, for instance:

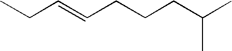

(wherein n is 2 and R⁹ and R¹⁰ are both H).

In the invention any of the groups defined as alkyl, aryl, alkaryl, aralkyl, heteroaryl, heterocyclyl, heteroaralkyl and heterocycloalkyl may be independently substituted on the backbone with one or more of the groups, preferably 1, 2, 3, 4, 5 or 6 groups, independently selected from C(O)OH, C(O)O(C₁₋₆ alkyl), C(O))(C₆₋₂₀ aryl), C(O)O(C₇₋₂₀ aralkyl), C(O)O(C₇₋₂₀ alkaryl), NHC(O)—CH═CH₂, —C≡C—H, halo, OH, O(C₁₋₆ alkyl), O(C₆₋₂₀ aryl), O(C₇₋₂₀ alkaryl), O(C₇₋₂₀ aralkyl), ═O, NH₂, ═NH, NH(C₁₋₆ alkyl), N(C₁₋₆ alkyl)₂, ═N(C₁₋₆ alkyl), NH(C₆₋₂₀ aryl), NH(C₇₋₂₀ alkaryl), NH(C₇₋₂₀ aralkyl), NHC(O)(C₁₋₆ alkyl), NHC(O)(C₆₋₂₀ aryl), NHC(O)(C₇₋₂₀ alkaryl), NHC(O)(C₇₋₂₀ aralkyl), NHC(O)(C₁₋₂₀ heteroaryl), NHC(O)(C₂₋₂₀ heterocyclyl), NHC(O)(C₂₋₂₀ heteroaralkyl), NHC(O)(C₃₋₂₀ heterocyclylalkyl), NHC(O)(C₃₋₂₀ alkylheterocyclyl), NO₂, CN, C(O)H, C(O)(C₁₋₆ alkyl), C(O)(C₆₋₂₀ aryl), C(O)(C₇₋₂₀ alkaryl), C(O)(C₇₋₂₀ aralkyl), C₁₋₁₀ alkyl, C₂₋₁₀ alkoxyalkyl, C₇₋₂₀ alkoxyaryl, C₁₂₋₂₀ aryloxyaryl, C₇₋₂₀ aryloxyalkyl, C₁₋₁₀ alkoxy, C₆₋₂₀ aryloxy, C₂₋₁₀ alkenyl, C₂₋₁₀ alkynyl, C₃₋₂₀ cycloalkyl, C₄₋₂₀ (cycloalkyl)alkyl, C₇₋₂₀ aralkyl, C₇₋₂₀ alkaryl, C₁₋₂₀ heteroaryl, epoxide and C₆₋₂₀ aryl.

Preferably, in the compound of general formula (I), R is a group of general formula (II).

A particularly preferred compound is

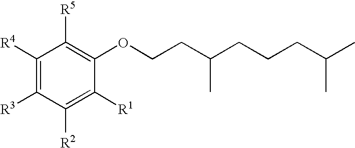

In the compound of general formula (I), at least one of R¹—R⁵ may be either a ketone or an imine.

In the compound according to the first aspect of this invention, R¹ is C(O)R⁸. In a preferred embodiment, R⁸ is C₁₋₂₀ alkyl. Even more preferably, R⁸ is C₁₋₅ alkyl, for instance, methyl, propyl or butyl. Most preferably, R⁸ is an isobutyl group.

R² and R⁴ are preferably selected from OH, OC₁₋₂₀ alkyl and OC₂₋₁₀ alkenyl.

Preferred groups for R³ and R⁵ are H, and C₁₋₁₀ alkyl.

In the compound according to the first aspect of this invention, R² and R⁴ are OR⁷. In one embodiment both R⁷s are hydrogen. However, in a different embodiment, only one of R² and R⁴ is OH, and the other is a group of formula OR⁷, wherein R⁷ is a group of general formula (II). Typically, R² is OH and R⁴ is OR⁷, wherein R⁷ is a group of general formula (II).

Preferably, the compound of general formula (I) comprises at least one group of general formula (II).

In a particularly preferred class of compounds of this invention, R³ and R⁵ are both H. More preferably, R³ and R⁵ are H, and R² and R⁴ are OR⁷, wherein preferably, R⁷ is H or a group of general formula (II).

In the group of general formula (II), preferably n is in the range 1 to 5. Most preferably, n is 2, 3 or 4. These represent monoterpenoids, sesquiterpenoids and diterpenoids respectively.

A particularly preferred compound of this invention has formula:

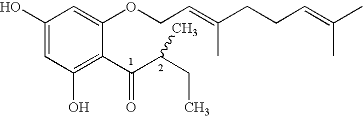

A particular stereochemistry is desired. The substituent R¹ preferably has the (S) stereochemistry at the 2-carbon. This is shown below for the preferred compound illustrated above.

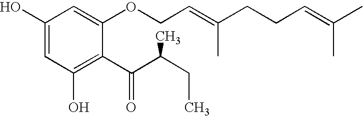

A preferred compound with two terpene substituents is shown below.

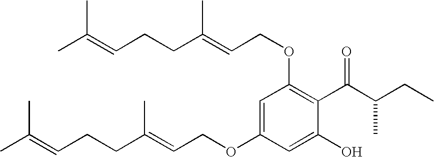

The terpene substituents may be saturated or un-saturated. For instance, group of formula (II) may have structure:

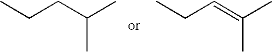

The compounds of this invention have been shown to have activity against Gram-positive bacteria, for instance multidrug-resistant strains of bacteria. Accordingly, the compounds of this invention may be incorporated into pharmaceutical compositions. The compounds of the invention may be prepared in racemic form or prepared in individual enantiomeric form by a specific synthesis or resolution as will be appreciated by the person skilled in the art.

A compound of the invention may be in a protected form.

In therapeutic use, the active compound may be administered orally, intravenously, rectally, parenterally, by inhalation, topically, ocularly, nasally or to the buccal cavity. Thus, the composition of the present invention may take the form of any known pharmaceutical compositions for such methods of administration. The compositions of the invention may contain 0.1 to 99% by weight of active compound. The compositions of the invention are generally preferred in unit dosage form. Preferably, a unit dose comprises the active ingredient in an amount 1 to 500 mg. The excipient used in the preparation of these compositions are the excipients known in the art.

Compositions for oral administration include known pharmaceutical forms for such administration, for example, tablets, lozenges, aqueous or oil suspensions, dispersible powders or granules, emulsions or syrups. The compositions may contain one or more agents such as sweetening agents, flavouring agents, colouring agents and preserving agents, in order to provide pharmaceutically elegant and palatable preparations. Tablets contain the active ingredient in admix with non-toxic pharmaceutically acceptable excipients which are suitable for the manufacture of tablets. These excipients may be, for instance, inert diluents such as calcium carbonate; granulating and disintegrating agents, for example corn starch, binding agents, for example starch gelatine; and lubricating agents, for example, magnesium stearate or talc.

The pharmaceutical composition may also be in the form of a sterile injectable aqueous or oleaginous suspension. This suspension may be formulated according to a known art using a suitable dispersing or wetting agents and suspending agents known in the art. The sterile injectable preparation may also be in a sterile injectable solution or suspension in a non-toxic parenterally acceptable diluent or solvent, for example as a solution in 1,3-butanediol. Among the acceptable vehicles and solvents that may be employed or water, Ringer's solution and isotonic sodium chloride solution. In addition, sterile, fixed oils are conventionally employed as a solvent or suspending medium.

The compound according to this invention may find use in therapy. In particular, the compound may be useful in the treatment of any bacterial infection. For instance, the compounds may have utility in the treatment of Gram-positive infections, such as S. aureus and Mycobacterium infections. The compounds may have utility against bacteria resistant to β-lactams, macrolides, fluoroquinolones and tetracyclines, particularly against infections by MRSA, penicillin and Mycobacterium tuberculosis resistant variants.

The compound may also be used in the treatment of infections caused by Staphylococcus epidermidis, Staphylococcus haemolyticus, Enterococcus faecium, Enterococcus faecalis, Streptococcus pneumoniae and Group A Streptococcus.

Methods for isolating the compound according to the first aspect of this invention from its natural source also form part of this invention. In a typical method of isolation, the plant suspected of containing the compound is pulverised together with one or more organic solvents to form one or more plant extracts, and the extract(s) are then purified to obtain the compound. Typically, compounds of this invention will be obtainable from the Hypericum genus of plants, in particular the species Hypericum perforatum, Hypericum papuanum, Hypericum beanii and other plant species such Helichrysum caespititium and Myrtus communis may also be used to extract the novel compounds of this invention.

By “pulverisation”, we mean a mechanical grinding action which reduces the particle size of the plant. Suitable means for this step are known in the art. In its most simplest form, a pestle and mortar may be used.

Typically, the compounds are isolated from aerial parts of the plant. Hexane and dichloromethane are suitable organic solvents for the extraction. Purification methods include chromatography, for instance, vacuum liquid chromatography or thin layer chromatography. Further details of one embodiment of the extraction process are described in Example 2.

The present invention also encompasses a plant extract comprising a compound according to the first aspect of this invention and one or more diluents. The diluents may be any solvent which is capable of dissolving the compound. For instance, the diluent may be an organic solvent such as hexane or dichloromethane. The plant extract may also comprise other compounds co-extracted with the compound according to the first aspect of the invention. These compounds may be further compounds of general formula (I) or alternatively, compounds of different structures which fall outside the scope of compounds of general formula (I), but which may also have a therapeutic effect.

The final aspect of this invention is directed to a method for chemically synthesising a novel compound of the invention. The preferred compounds which are synthesised have the same features as the preferred compounds according to the first aspect of the invention. More particularly, the method is directed to a method of synthesising a compound of general formula (VII):

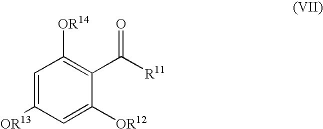

In this embodiment, the method comprises the steps of:

(i) reacting phloroglucinol in a Friedel-Crafts acylation with a compound of formula (V)

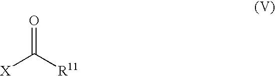

wherein X is Cl or Br and R¹¹ is C₁₋₂₀ alkyl, H, C₂₋₁₀ alkoxyalkyl, C₇₋₂₀ alkoxyaryl, C₁₂₋₂₀ aryloxyaryl, C₇₋₂₀ aryloxyalkyl, C₁₋₁₀ alkoxy, C₆₋₂₀ aryloxy, C₂₋₁₀ alkenyl, C₂₋₁₀ alkynyl, C₃₋₂₀ cycloalkyl, C₄₋₂₀ (cycloalkyl)alkyl, C₇₋₂₀ aralkyl, C₇₋₂₀ alkaryl, C₁₋₂₀ heteroary, C₆₋₂₀ aryl, OR⁶, NHR⁶ or N(C₁₋₆ alkyl)₂;

wherein R⁶ is as defined previously;

to give a compound of general formula (VI):

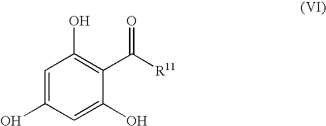

(ii) optionally protecting one or more hydroxyl groups to give an optionally protected compound;

(iii) reacting the optionally protected compound produced in step (ii) with a compound of general formula (IV)

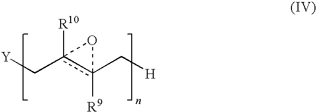

wherein n is 1-5, Y is Cl, Br or I, and R⁹ and R¹⁹ are as defined previously; and de-protecting, if necessary, to give a compound of general formula (VII)

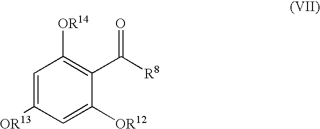

wherein at least one of R¹²—R¹⁴is a group of general formula (VIII)

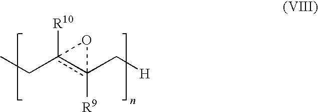

and the remainder are H or C₁₋₁₀ alkyl.

The compound thus synthesised has at least one terpene substituent. As is well known in the art, a starting material for monoterpenes is a geranyl halide, such as geranyl bromide. Similarly, farnesyl bromide is a typical starting point for sesquiterpenes and geranyl geranyl bromide is a typical starting point for diterpenes.

After reaction with the compound of general formula (VI), the terpene-substituent may be subjected to further reaction steps. For instance, if the terpene substituent is unsaturated, one or more of the double bonds may be reduced with hydrogen. Reduction conditions are well known to those skilled in the art, and include gaseous H₂ over a Nickel catalyst.

In the method, in compound of formula (IV) preferably n is 2, 3 or 4.

The hydroxyl group on the phenolic ring may be protected by any suitable phenolic hydroxyl protecting group known in the art. A silyl ether protecting group is preferred, such as TBDPS. A benzylether may also be used. One or more of the hydroxyl groups on the phenol ring may be protected. In a particularly preferred embodiment, only two of the hydroxyl groups are protected to give a compound of general formula (IX)

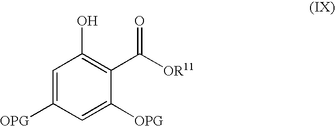

In this structure PG is the hydroxyl protecting group.

When two hydroxyl groups are protected in this manner, the compound of general formula VII produced (on completion of the synthesis) is generally

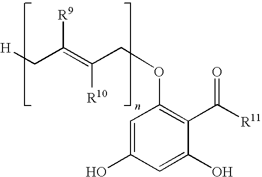

When protecting group chemistry is not used, the typical product, i.e. compound of general formula VII is:

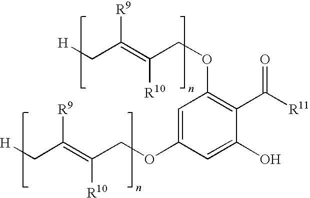

In these compounds, R⁹, R¹⁰ and n are as defined previously. Each R⁹, R¹⁰ and n may be the same or different.

In some embodiments of the invention, particularly when R¹¹ is not a sterically bulky group, the H of all phenolic OH groups may be derivatised with a group of formula (II).

For the Friedel Crafts reaction, suitable conditions are well known in the art. AlCl₃ is typically used as a catalyst in the presence of the carbon disulfide in a solvent such as nitrobenzene.

Step (iii) may suitably be carried out in the presence of a base such as potassium carbonate and dimethyl formamide. Such reaction conditions typically promote simultaneous cleavage of the protecting groups, if present.

In the method according to the final aspect of this invention, preferably R¹¹ is C₁₋₂₀ alkyl, more preferably C₁₋₁₀ alkyl. In a particularly preferred embodiment R¹¹ is 2-(S)-methyl propyl group.

The invention will be now illustrated by the following non-limiting Examples.

### Example 1

### Synthesis of alkylated 2-acylphloroglucinols

2-(2-methylbutanoyl)phloroglucinol (alternatively 2-methyl-1-(2,4,6-trihydroxyphenyl) butan-1-one; 4) is prepared by Friedel-Crafts acylation of phloroglucinol (3) using 2-methylbutanoyl chloride (2) in the presence of aluminium trichloride and carbon disulfide in nitrobenzene. 2-Methylbutanoyl chloride is commercially available as a racaemic mixture, giving a racaemic product. The commercially available (S)-enantiomer of 2-methylbutanoic acid 1 can be converted to the corresponding acid chloride by refluxing in thionyl chloride, followed by isolation by distillation. The (S)-2-methylbutanoyl chloride 2 is then utilised in the same manner to produce the corresponding (S)-enantiomer of the 2-acylphloroglucinol (4) stereoselectively.

For the conversion of 1-S-2-methylbutyric acid→S-2-methylbutyryl chloride, the reaction conditions are as follows:

### Reagents:

S-2-methylbutyric acid, 10 g (97.91 mmol)
Thionyl chloride, 1.5 Eq, 10.71 mL

### Product:

S-2-methylbutyryl chloride, 10.63 g (88.21 mmol, 90.1% yield)

### Conditions:

Reflux for 2 hrs at 80° C.
The product is purified by distillation. B.p. of the product is 119-120° C., b.p. for the starting material is 78-80° C.
For the Friedel-Crafts acylation, the reaction conditions are as follows:

### Reagents:

S-2-methylbutyryl chloride, 10.34 g (85.8 mmol)
Phloroglucinol, 1 Eq, 10.81 g
Anhydrous AICl₃, 4.1 Eq, 46.43 g
Carbon disulphide, 50 mL
Nitrobenzene, 45 mL

### Product:

Acylphloroglucinol, 9.71 g (46.19 mmol, 53.8% yield)

### Conditions:

AlCl₃ was added to a suspension of phloroglucinol in carbon disulphide.
Nitrobenzene (40 mL) was then added over 30 min.
The solution was heated under reflux for 30 min at 55° C.
The acyl chloride was dissolved in nitrobenzene (5 mL) and then added over 30 min.

Following acylation, the 2-(2-methylbutanoyl)phloroglucinol 4 is regioselectively protected in good yield in the para-position and one ortho-position as the bis-tert-butyldiphenylsilyl ether 5, using tert-butyldiphenylchlorosilane in the presence of imidazole in acetone. The reaction conditions are as follows:

### Reagents:

Acylphloroglucinol, 9.71 g (46.19 mmol)
Imidazole, 3 Eq, 9.43 g
TBDMS-Cl, 2.1 Eq, 14.61 g

### Product:

Protected acylphloroglucinol, 16.4 g (37.26 mmol, 80.7% yield)

### Conditions:

The mixture was stirred in acetone for 2 hours at room temperature.

Finally, the remaining phenolic hydroxyl group (ortho to the ketone) is alkylated using geranyl bromide in the presence of potassium carbonate in N,N-dimethylformamide, with simultaneous cleavage of both silyl ethers in one pot, to afford the desired 1-(2-geranyloxy-4,6-dihydroxyphenyl)butan-1-one 6. The reaction conditions for alkylation and deprotection are as follows:

### Reagents:

Protected acylphloroglucinol, 6.6 g (15.0 mmol)
Geranyl Br, 1.2 Eq, 3.43 mL
Anhydrous K₂CO₃, 1.5 Eq, 3.1 g
Dried DMF 100 mL

### Product:

The required natural product, 220 mg (0.636 mmol, 4.2%)

### Conditions:

The mixture was stirred in DMF at 80° C. for 3 hours.
   Notes on reaction 4—When the reaction was carried out in a small scale (i.e. 29 mg of protected acylphloroglucinol), the yield of the product was 46.0%, giving 10.5 mg of product.

The (S)-enantiomer is prepared starting from (S)-2-methyl-1-(2,4,6-trihydroxyphenyl)butan-1-one.

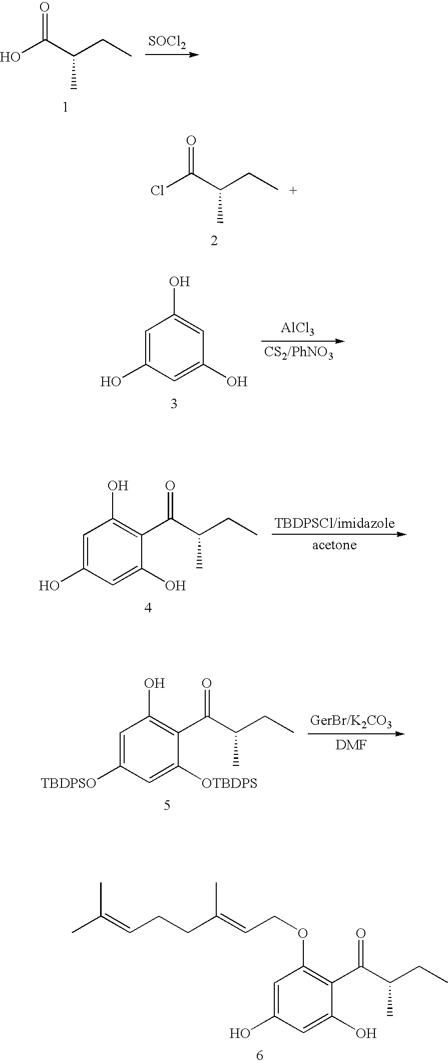

### Example 2

Compound 7 (see structure below) was isolated from the hexane and dichloromethane extracts of the aerial parts of Hypericum olympicum cf uniflorum collected from the Royal Botanic Gardens at Wakehurst place (accession number 1969-31184).

### Example 2.1

The hexane extract was subjected to vacuum-liquid chromatography on silica gel eluting with hexane containing 10% increments of ethyl acetate to yield 12 fractions. The fraction eluted with 60% ethyl acetate was further separated by Sephadex LH20 chromatography eluted with chloroform and methanol (1:1) yielding 6 fractions. Fraction 6 was purified by preparative thin-layer chromatography (pTLC) using toluene-ethyl acetate-acetic acid (80:18:2), yielding compound 7.

### Example 2.2

The dichloromethane extract was subjected to Sephadex LH20 chromatography eluted with chloroform and methanol (1:1) yielding 6 fractions. Fractions 4 to 6 were further purified by pTLC using toluene-ethyl acetate-acetic acid (75:23:2), yielding compound 7.

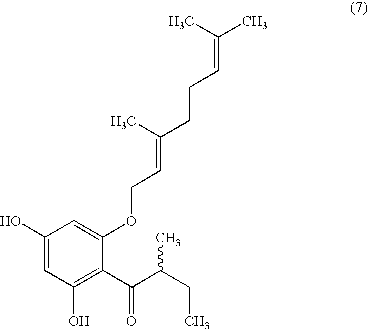

### Example 3

### Structure elucidation of compound 7

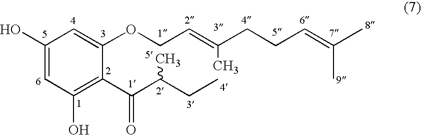

HR ESI-TOF-MS suggested a molecular formula of C₂₁H₃₀O₄ [M−H]⁻ (345.2056). The ¹H NMR spectrum (Table 1) showed two signals for hydroxyl groups, one of which was highly deshielded hydrogen-bonded (δ_{H} 14.02) and the other appeared as a broad singlet at δ_{H} 5.32. Other signals observed in the ¹H NMR spectrum included two meta-coupled aromatic protons (δ_{H} 5.98 d, J=2.5; 5.92 d, J=2.5), two olefinic protons (δ_{H} 5.51 m, 1H; 5.10 m), one methine (δ_{H} 3.66, 1H), four methylene groups, three methyl singlets (δ_{H} 1.74, 1.69, 1.62), one methyl doublet (δ_{H} 1.12, J=6.5) and one methyl triplet (δ_{H} 0.89, J=7.5). The ¹³C spectrum (Table 1) displayed signals for six aromatic carbons, three of which were highly deshielded, implying that these carbons were attached to electron-withdrawing groups. The pattern of these signals suggested a 1,3,5-trihydroxybenzene (phloroglucinol) structure. In the HMBC spectrum the hydrogen-bonded proton showed ²J correlation with the carbon to which it was directly attached (δ_{C} 167.5, C-1), and ³J correlations with an aromatic carbon attached to a proton (δ_{C} 96.5, C-6) and a quaternary aromatic carbon (δ_{C} 105.0, C-2), confirming the position of this hydroxyl group. The other aromatic proton at δ_{H} 5.92 was then placed at C-4 as it was meta-coupled. This was further confirmed by HMBC correlations between this proton and C-2, C-6 and a deshielded carbon (δ_{C} 161.9, C-5). The second hydroxyl group was therefore placed between the aromatic protons at C-5.

The oxymethylene group (δ_{H} 4.57 d, J=6.5) showed ¹H-¹³C correlations with the remaining deshielded aromatic carbon (δ_{C} 162.6, C-3) and two carbons associated with an olefin group (δ_{C} 118.2, C-2″; δ_{C} 142.3, C-3″). The olefinic proton (δ_{H} 5.51 m) at C-2″ was coupled to a methyl group (δ_{C} 16.7, C-10″) and a methylene group (δ_{C} 39.5, C-4″) via three bonds. The protons of this methylene group (δ_{H} 2.13 m) showed ²J correlations with C-3″ and a further methylene group (δ_{C} 26.3, C-5″), and ³J correalations with C-2″ and a further olefinic carbon (δ_{C} 123.6, C6″). The two methyl singlets (δ_{H} 1.62, 1.69) which coupled to C6″ and a quaternary carbon associated with this olefin (δ_{C} 132.0, C-7″) completed the substituent at C-3. This side-chain consisted of 10 carbons, two olefin groups and three methyl groups, which is characteristic of a geranyl group. The COSY and NOESY spectra also provided evidence for the geranyl side-chain. Both olefinic protons and olefinic methyls were assigned as the trans configuration on biosynthetic grounds.

The final substituent at position 2 included a methine multiplet (δ_{H} 3.66, H-2′), a methylene multiplet (δ_{H} 1.37, 1.80, H-3′), a methyl triplet (δ_{H} 0.89, H-4′) and a methyl doublet (δ_{H} 1.12, H-5′). In the COSY spectrum, the methylene was coupled to the methyl triplet and the methine multiplet which was coupled to the methyl doublet. HMBC correlations showed crosspeaks between the methyl doublet and C-2″, C3″ and a carbonyl carbon (δ_{C} 210.4, C-1″) which could interact with the hydroxyl group at C-1 via hydrogen bond. This confirmed the 2-methylbutanoyl side-chain at position 2 and completed the structure elucidation of 7. Compound 7 was therefore identified as 1,5-dihydroxy-2-(2′-methylbutanoyl)-3-(3″,7″-dimethyl-2″,6″-octadienyl)-benzene. This is a new natural product and is reported here for the first time.

We have recently synthesised this molecule and the NMR data of the natural and synthetic material is identical.

TABLE 1

| | | | | |
|---|---|---|---|---|
| Position | ¹H | ¹³C | ²J | ³J |

| 1 | — | | 167.5 | — | — |
|---|---|---|---|---|---|
| 2 | — | | 105.0 | — | — |
| 3 | — | | 162.6 | — | — |
| 4 | 5.92 | d (2.5) | 91.5 | C5 | C2, C6 |
| 5 | — | | 161.9 | — | — |
| 6 | 5.98 | d (2.5) | 96.5 | C1 | C2, C4 |
| 1′ | — | | 210.4 | — | — |
| 2′ | 3.66 | m | 46.1 | — | — |
| 3′ | 1.37 m, 1.80 m | | 26.8 | — | — |
| 4′ | 0.89 | t (7.5) | 11.8 | C3′ | C2′ |
| 5′ | 1.12 | d (6.5) | 16.6 | C2′ | C1′, C3′ |
| 1″ | 4.57 | d (6.5) | 65.7 | C2″ | C3, C3″ |
| 2″ | 5.51 | m | 118.2 | — | C4″, C10″ |
| 3″ | — | | 142.3 | — | — |
| 4″ | 2.13 | m | 39.5 | C3″, C5″ | C2″, C6″ |
| 5″ | 2.10 | m | 26.3 | C4″, C6″ | C3″ |
| 6″ | 5.10 | m | 123.6 | — | — |
| 7″ | — | | 132.0 | — | — |
| 8″ | 1.62 | s | 17.7 | C7″ | C6″, C9″ |
| 9″ | 1.69 | s | 25.7 | C7″ | C6″, C8″ |
| 10″ | 1.74 | s | 16.7 | C3″ | C2″, C4″ |
| 5-OH | 5.32 | bs | — | — | — |
| 1-OH | 14.02 | s | — | C1 | C2, C6 |

¹H (500 MHz) and ¹³C NMR (125 MHz) spectral data and ¹H-¹³C long-range correlations of 7 recorded in CDCl₃

### Example 4

### Minimum inhibitory concentration (MIC) assay

Bacteria were cultured on nutrient agar (Oxoid) and incubated for 24 h at 37 ° C. prior to MIC determination. The control antibiotics norfloxacin, tetracycline and erythromycin were obtained from Sigma Chemical Co. Mueller-Hinton broth (MHB; Oxoid) was adjusted to contain 20 and 10 mg/l of Ca²⁺ and Mg²⁺, respectively. An inoculum density of 5×10⁵ cfu of S. aureus was prepared in normal saline (9 g/l) by comparison with a 0.5 MacFarland turbidity standard. The inoculum (125 μl) was added to all wells and the microtitre plate was incubated at 37° C. for 18 h. For MIC determination, 20 μl of a 5 mg/ml methanolic solution of 3-[4,5-dimethylthiazol-2-yl]-2,5-diphenyltetrazolium bromide (MTT; Sigma) was added to each of the wells and incubated for 20 min. Bacterial growth was indicated by a colour change from yellow to dark blue. The MIC was recorded as the lowest concentration at which no growth was observed. Table 2 shows the results.

TABLE 2
MICs of standard antibiotics and active compound 7

| | | | |
|---|---|---|---|
| | | MIC of | MIC of |
| | Standard | antibiotic | compound 7 |
| Strain | antibiotic(s) | (μg/ml) | (μg/ml) |

S. aureus

| | | | |
|---|---|---|---|
| SA1199B | Norfloxacin | 32 | 1 |
| XU212 | Tetracycline | 128 | 1 |
| RN4220 | Erythromycin | 128 | 1 |
| ATCC25923 | Norfloxacin | 1 | 1 |
| EMRSA15 | Oxacillin | 16 | 0.5 |
| EMRSA16 | Oxacillin | 512 | 1 |

Mycobacterium

| | | | |
|---|---|---|---|
| M. smegmatis ATCC14468 | Isoniazid; | 2; 0.5 | 4 |
| | Ethambutol | | |
| M. fortuitum ATCC6841 | Isoniazid; | 4; 0.5 | 8 |
| | Ethambutol | | |
| M. smegmatis MC2 2700 | Isoniazid; | 2; 0.5 | 4 |
| | Ethambutol | | |
| M. phlei ATCC11758 | Isoniazid; | 2; 0.5 | 4 |
| | Ethambutol | | |

P. aeruginosa

| | | | |
|---|---|---|---|
| K1119 | Norfloxacin | 2 | Not active at |
| | | | 512 μg/ml |
| K767 | Norfloxacin | 2 | Not active at |
| | | | 512 μg/ml |

S. typhimurium

| | | | |
|---|---|---|---|
| L354 | Tetracycline | 1* | Not active at |
| | | | 512 μg/ml |
| L10 | Tetracycline | 1* | Not active at |
| | | | 512 μg/ml |

### Example 5

Compound 7 was tested using bacteria which were recent clinical isolates, or were standard reference controls. The clinical isolates were selected to represent important resistant phenotypes currently prevalent in the UK and worldwide. They included (i) methicillin-resistant Staphylococcus aureus (MRSA), specifically the epidemic (E)MRSA-15 and 16 strains dominant in the UK; (ii) methicillin-resistant coagulase negative staphylococci (i.e. S. epidermidis, S. haemolyticus); (iii) vancomycin-resistant as well as susceptible Enterococcus faecalis and E. faecium; (iv) penicillin-resistant and—susceptible Streptococcus pneumoniae; (v) group A streptococci (=S. pyogenes, which remain universally susceptible to penicillin) and (vi) Clostridium difficile.

MICs were determined on IsoSensitest agar (ISA) (method of British Society for Antimicrobial Chemotherapy, http://www.bsac.org.uk) or by microdilution in IsoSensitest Broth (ISB). Both media were from Oxoid, Basingstoke, Hants. Plates were variously incubated, at 35-37° C., in air, air enriched with 5% CO₂ or under anaerobic conditions.

Results are as follows and activities as minimum inhibitory concentration (in μg/ml) in broth and agar are given in parentheses after the bacterial strains: epidemic methicillin-resistant Staphylococcus aureus 15 and 16 (2-4 μg/ml), methicillin-sensitive Staphylococcus aureus (2-8 μg/ml), methicillin-resistant coagulase-negative Staphylococcus epidermidis (2-8 μg/ml), methicillin-sensitive coagulase-negative Staphylococcus epidermidis (2-4 μg/ml), methicillin-sensitive coagulase-negative Staphylococcus haemolyticus (16 μg/ml), vancomycin-resistant and vancomycin-sensitive Enterococcus faecium (2-4 μg/ml), vancomycin-resistant and vancomycin-sensitive Enterococcus faecalis (4 μg/ml), penicillin-resistant and penicillin-sensitive Streptococcus pneumoniae (2-4 μg/ml), Group A Streptococcus (4 μg/ml), and standard laboratory strains Streptococcus pneumoniae ATCC 29212 (4 μg/ml), Enterococcus faecalis ATCC29212 (4 μg/ml), NCTC6571 Staphylococcus aureus (4 μg/ml).

1-34. (canceled)

35. A compound of general formula (I) 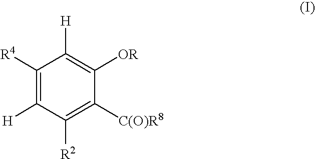
wherein R is selected from C₁₋₂₀ alkyl, C₂₋₂₀ alkoxyalkyl, C₂₋₂₀ alkenyl, C₂₋₂₀alkynyl, C₃₋₂₀ cycloalkyl and C₄₋₂₀ (cycloalkyl)alkyl;
R² and R⁴ are each independently selected from OR⁷;
wherein each R⁷ may be the same or different and is selected from C¹⁻²⁰ alkyl, H, C₂₋₁₀ alkoxyalkyl, C₇₋₂₀ alkoxyaryl, C₁₂₋₂₀ aryloxyaryl , C₇₋₂₀aryloxyalkyl, C₂₋₁₀ alkenyl, C₂₋₁₀ alkynyl, C₃₋₂₀ cycloalkyl, C,₄₋₂₀ (cycloalkyl)alkyl, C₇₋₂₀ aralkyl, C₇₋₂₀ alkaryl, C₁₋₂₀ heteroaryl, C₆₋₂₀ aryl, C(O)H, C(O)OH, C(O)R⁸, C(O)OR⁶, C(O)NH₂, C(O)NR⁶H and C(O)N(C₁₋₆alkyl)₂; and
wherein R⁸ is C₂₋₂₀ alkyl;
wherein any of the groups alkyl, aryl, alkaryl, aralkyl, heteroaryl, eterocyclyl, heteroaralkyl and heterocycloalkyl may be independently substituted on the backbone with one or more of the groups, preferably 1, 2, 3, 4, 5 or 6 groups, independently selected from C(O)OH, C(O)O(C₁₋₆ alkyl), C(O)O(C₆₋₂₀ aryl), C(O)O(C₇₋₂₀aralkyl), C(O)O(C₇₋₂₀ alkaryl), NHC(O)—CH═CH₂, —C═C—H, halo, OH, O(C₁₋₆ alkyl), O(C₆₋₂₀ aryl), O(C₇₋₂₀ alkaryl), O(C₇₋₂₀ aralkyl), O, NH₂, ═NH, NH(C₁₋₆ alkyl), N(C₁₋₆ alkyl)2, ═N(C₁₋₆ alkyl), NH(C₆₋₂₀ aryl), NH(C₇₋₂₀ alkaryl), NH(C₇₋₂₀ aralkyl), NHC(O)(C₁₋₆ alkyl), NHC(O)(C₆₋₂₀ aryl), NHC(O) (C₇₋₂₀ alkaryl), NHC(O)(C₇₋₂₀ aralkyl), NHC(O)(C₁₋₂₀heteroaryl), NHC(O)(C₂₋₂₀heterocyclyl), NHC(O)(C₂₋₂₀heteroaralkyl), —NHC(O)(C₃₋₂₀ heterocyclylalkyl), NHC(O)(C₃₋₂₀ alkylheterocyclyl), NO2, CN, C(O)H, C(O)(C_{1 -6} alkyl), C(O)(C₆₋₂₀ aryl), C(O)(C₇₋₂₀ alkaryl), C(O)(C₇₋₂₀ aralkyl), C₁₋₁₀ alkyl, C₂₋₁₀ alkoxyalkyl, C₇₋₂₀ alkoxyaryl, C₁₂₋₂₀ aryloxyaryl, C₇₋₂₀ aryloxyalkyl, C₁₋₁₀ alkoxy, C₆₋₂₀ aryloxy, C₂₋₁₀ alkenyl, C₂₋₁₀ alkynyl, C₃₋₂₀ cycloalkyl, C₄₋₂₀ (cycloalkyl)alkyl, C₇₋₂₀ aralkyl, C_{7—}7₀ alkaryl, C₁₋₂₀ heteroaryl, epoxide and C₆₋₂₀ aryl.

36. A compound according to claim 35, wherein R is a group of formula (II) 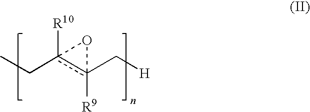
wherein R⁹ and R¹⁰ are independently H or C₁₋₁₀ alkyl and n is 1-5.

37. A compound according to claim 35, wherein R is C₁₋₂₀ alkyl, preferably C₂₋₂₀ alkyl, most preferably C₃₋₂₀ alkyl.

38. A compound according to claim 36, wherein R is C₁₋₂₀ alkyl, preferably C₂₋₂₀ alkyl, most preferably C₃₋₂₄) alkyl.

39. A compound according to claim 35, of formula 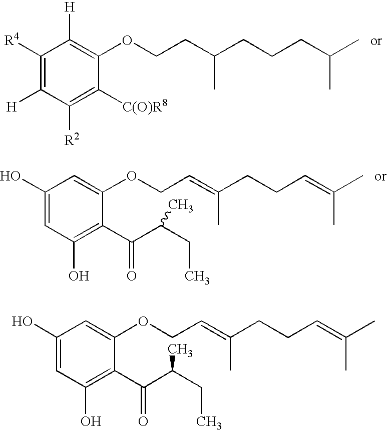
wherein R², R⁴, R⁷ and R⁸ are as defined in claim 35.

40. A compound according to claim 35, wherein R⁸ is a 1-methyl propyl group.

41. A compound according to claim 35, wherein each R⁷ is H.

42. A compound according to claim 35, wherein R² is OH and R⁴ is OR⁷, wherein R⁷ is a group of formula (II) 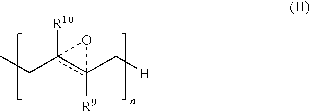
wherein R⁹ and R¹⁰ are independently H or C₁₋₁₀ alkyl and n is 1-5.

43. A compound according to claim 36, wherein n is 2.

44. A compound according to claim 36, wherein R⁹ is methyl.

45. A compound according to claim 36, wherein R¹⁰ is H.

46. A pharmaceutical composition comprising a compound according to claim 35, and one or more pharmaceutically acceptable excipients.

47. A method of inhibiting bacterial growth or proliferation comprising contacting a bacteria with a growth inhibiting or proliferation inhibiting amount of a compound according to claim 35.

48. A method of inhibiting a bacterial infection in a subject comprising administering an antibacterial effective amount of a compound according to claim 35 to a subject having a bacterial infection, whereby the bacterial infection is inhibited.

49. The method of claim 48, wherein the infection is by a Gram-positive bacteria.

50. The method of claim 49, wherein the Gram-positive bacteria is methicillin-resistant or penicillin resistant Staphylococous aureus, multi-drug resistant S. aureus, or Mycobacterium tuberculosis.
